(19) United States
(12) Patent Application Publication
(10) Pub. No.: US 2025/0288347 A1
(43) Pub. Date: Sep. 18, 2025

(54) ENDOSCOPIC TREATMENT INSTRUMENT

(71) Applicant: OLYMPUS MEDICAL SYSTEMS CORP., Tokyo (JP)

(72) Inventors: Kazuki OZAWA, Hachioji-shi (JP); Shunsuke Motosugi, Hachioji-shi (JP); Yuta Muyari, Hino-shi (JP); Yohisane Nakamura, Hachioji-shi (JP); Hiromasa Kato, Tokyo (JP)

(21) Appl. No.: 19/061,966

(22) Filed: Feb. 24, 2025

Related U.S. Application Data
(60) Provisional application No. 63/564,938, filed on Mar. 13, 2024.

(51) Int. Cl. A61B 18/14; A61B 18/00

(52) U.S. Cl. CPC A61B 18/14; A61B 2018/00178; A61B 2018/00184; A61B 2018/0091; A61B 2018/00982

(57) ABSTRACT

An endoscopic treatment instrument includes: a sheath; an electrode which is disposed on a distal end side of the sheath; a rod which is disposed at least partially inside the electrode; and an elastic body which biases a distal end of the rod so that the distal end of the rod is accommodated in more proximal side than a distal end of the electrode.

### CROSS-REFERENCE TO RELATED APPLICATIONS

Priority is claimed on U.S. Provisional Application No. 63/564,938, filed Mar. 13, 2024, the content of which is incorporated herein by reference.

### FIELD

The present disclosure relates to an endoscopic treatment instrument.

### BACKGROUND

Conventionally, in endoscopic treatment such as endoscopic submucosal dissection (ESD), endoscopic treatment instruments for incision and dissection, such as high-frequency knives, and endoscopic treatment instruments for local injection, etc. have been used.

An endoscopic treatment instrument described in Japanese Unexamined Patent Application, First Publication No. 2024-091534 (which is referred to as Patent Document 1) includes a rod that is advanceable and retractable relative to an electrode, and can perform incision and dissection treatment and local injection treatment.

### SUMMARY

However, the endoscopic treatment instrument described in Patent Document 1 requires a surgeon to intentionally change the operation of advancing and retracting the electrode and the operation of advancing and retracting the rod depending on the treatment situation, and it is necessary to make these operations easier.

The present disclosure provides an endoscopic treatment instrument with an electrode and a rod that are easily operated depending on the treatment situation, such as incision and dissection treatment and local injection treatment.

An endoscopic treatment instrument according to a first aspect of the present disclosure includes: a sheath; an electrode which is disposed on a distal end side of the sheath; a rod which is disposed at least partially inside the electrode; and an elastic body which biases a distal end of the rod so that the distal end of the rod is accommodated in more proximal side than a distal end of the electrode.

The electrode and the rod of the endoscopic treatment instrument of the present disclosure are easily operated depending on the treatment situation, such as incision and dissection treatment and local injection treatment.

### BRIEF DESCRIPTION OF THE DRAWINGS

FIG. 1 is an overall view of an endoscopic treatment system of a first embodiment.

FIG. 2 is an overall view showing a treatment instrument of the endoscopic treatment system.

FIG. 3 is a perspective view of a distal end portion of the treatment instrument.

FIG. 4 is a cross-sectional view of the distal end portion of the treatment instrument.

FIG. 5 is same as above.

FIG. 6 is a cross-sectional view of the distal end portion of the treatment instrument when a rod is in a third position.

FIG. 7 is a cross-sectional view taken along line X1-X1 shown in FIG. 6.

FIG. 8 is a cross-sectional view of an operation member and an operation portion.

FIG. 9 is a diagram illustrating the operation of the treatment instrument.

FIG. 10 is same as above.

FIG. 11 is same as above.

FIG. 12 is same as above.

FIG. 13 is a diagram showing the rod puncturing tissue.

FIG. 14 is a diagram showing a knife for delivering liquid into tissue.

FIG. 15 is a diagram showing a modified example of an elastic body.

FIG. 16 is a diagram showing a modified example of a lever.

FIG. 17 is a cross-sectional view taken along line X2-X2 shown in FIG. 16.

FIG. 18 is a diagram showing a modified example of an operation portion.

FIG. 19 is same as above.

FIG. 20 is a diagram showing a modified example of a rod.

FIG. 21 is a diagram showing a modified example of a first connector of the treatment instrument.

FIG. 22 is a diagram showing another modified example of the first connector.

FIG. 23 is a diagram showing another modified example of the first connector.

FIG. 24 is a diagram showing another modified example of the first connector.

FIG. 25 is a diagram showing another modified example of the first connector.

FIG. 26 is a diagram showing a distal end portion of a modified example of the treatment instrument.

FIG. 27 is a diagram illustrating an operation of a treatment instrument of a second embodiment.

FIG. 28 is same as above.

FIG. 29 is same as above.

FIG. 30 is same as above.

FIG. 31 is a diagram illustrating an operation of a treatment instrument of a third embodiment.

FIG. 32 is same as above.

FIG. 33 is a diagram showing a modified example of a first connector of the treatment instrument.

FIG. 34 is a diagram showing another modified example of the first connector.

FIG. 35 is a diagram showing a modified example of the elastic body.

### DETAILED DESCRIPTION

### First Embodiment

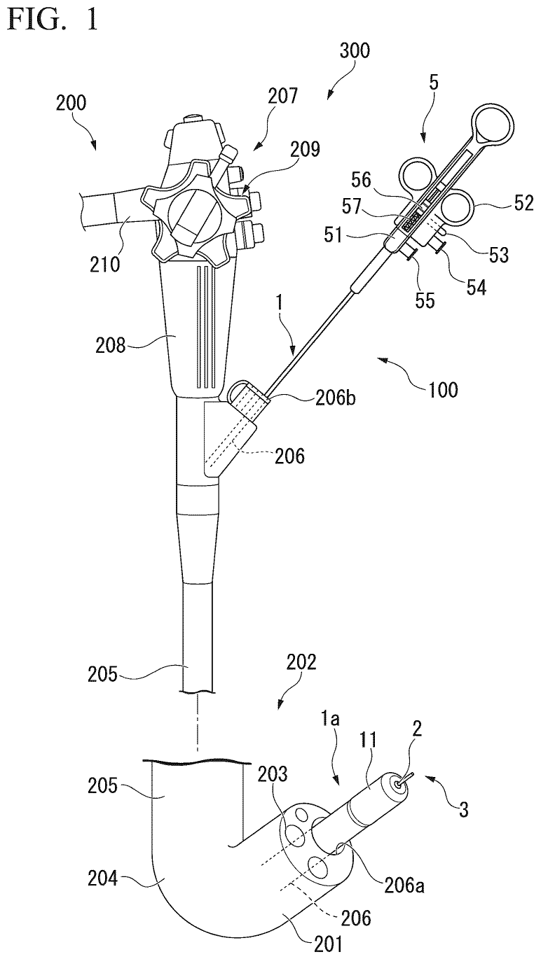

An endoscopic treatment system 300 according to a first embodiment of the present disclosure will be described with reference to FIGS. 1 to 12. FIG. 1 is an overall view of the endoscopic treatment system 300 according to this embodiment.

### Endoscopic Treatment System 300

The endoscopic treatment system 300 includes, as shown in FIG. 1, an endoscope 200 and a treatment instrument 100. The treatment instrument 100 is used by being inserted into the endoscope 200.

### Endoscope 200

The endoscope 200 is a well-known flexible endoscope, and includes an insertion portion 202 inserted into a body from a distal end, an operation portion 207 attached to a proximal end of the insertion portion 202, and a universal cord 210 attached to the operation portion 207.

The insertion portion 202 is a long thin member that can be inserted into a lumen. The insertion portion 202 has a distal end portion 201, a bending portion 204, and a flexible portion 205. The distal end portion 201, the bending portion 204, and the flexible portion 205 are connected in this order from the distal end side. A channel 206 for inserting the treatment instrument 100 is provided inside the insertion portion 202. The distal end portion 201 is provided with a distal end opening portion 206a of the channel 206 and an imaging unit 203.

The imaging unit 203 includes an imaging element such as a CCD or a CMOS, and is capable of capturing an image of a treatment site. The imaging unit 203 can capture an image of the distal end portion of the treatment instrument 100 in a state in which the treatment instrument 100 protrudes from the distal end opening portion 206a of the channel 206. The bending portion 204 bends according to an operator's operation of the operation portion 207. The flexible portion 205 is a tubular portion having flexibility.

The operation portion 207 is connected to the flexible portion 205. The operation portion 207 has a grip 208, an input unit 209, and a proximal end opening portion 206b of the channel 206. The grip 208 is a portion that is held by the operator. The input unit 209 receives an operation input for performing a bending operation on the bending portion 204.

The universal cord 210 connects the endoscope 200 to an external device. An imaging cable, an optical fiber cable, or the like that outputs an imaging signal captured by the imaging unit 203 to the outside is inserted into the universal cord 210.

### Treatment Instrument 100

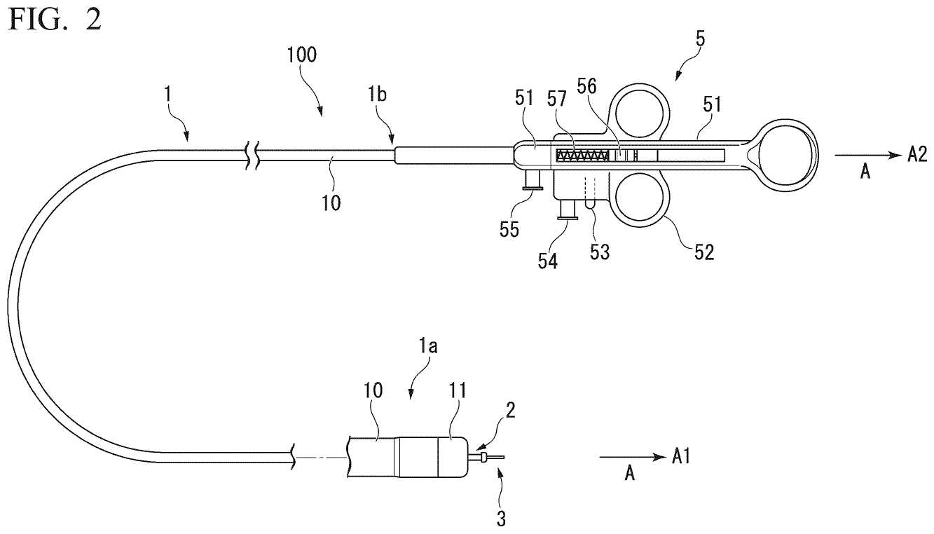

FIG. 2 is an overall view showing the treatment instrument 100.

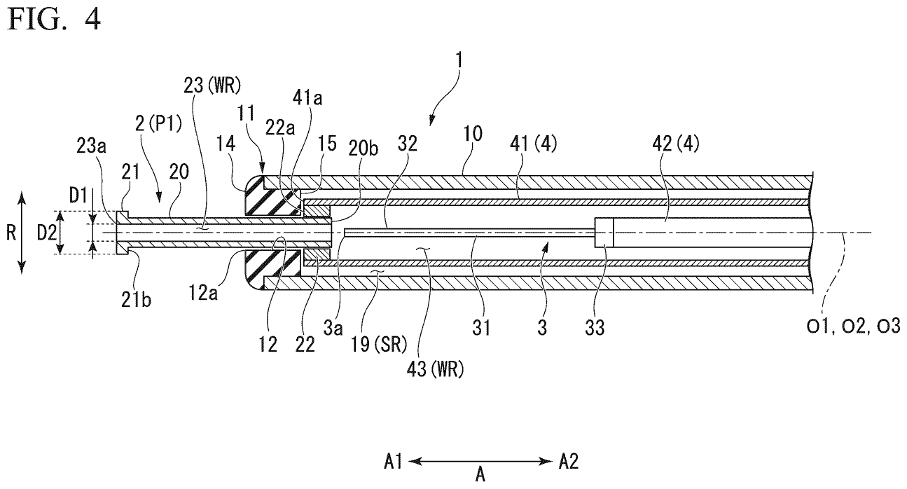

The treatment instrument (endoscopic treatment instrument) 100 includes an outer sheath 1, a knife 2, a rod 3, an operation member 4 (see FIG. 4), and an operation portion 5. In the following description, in the longitudinal direction (longitudinal axis direction, axial direction) A of the treatment instrument 100, the side inserted into the patient's body is referred to as the “distal end side (distal side) A1” and the side of the operation portion 5 is referred to as the “proximal end side (proximal side) A2.”

The outer sheath 1 is a long tubular member extending from a distal end la to a proximal end 1b. The outer sheath 1 can be inserted into the channel 206 of the endoscope 200 and can advance and retract through the channel 206. As shown in FIG. 1, the distal end la of the outer sheath 1 can protrude and retract from the distal end opening portion 206a of the channel 206 while the outer sheath 1 is inserted into the channel 206.

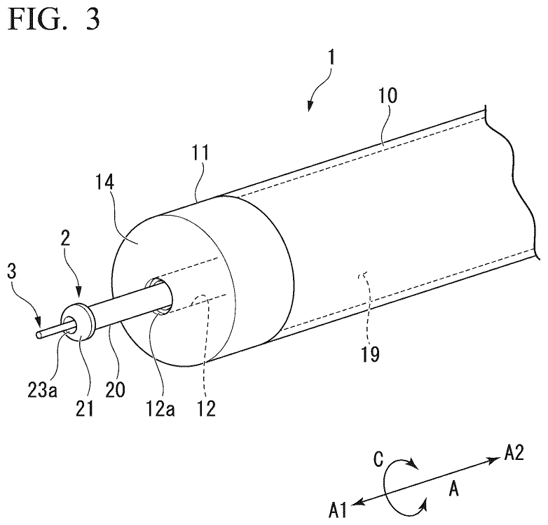

FIG. 3 is a perspective view of the distal end portion of the treatment instrument 100.

The outer sheath 1 has a tube 10 which extends in the longitudinal direction A and a distal end member 11 that is provided at the distal end of the tube 10. Furthermore, the outer sheath 1 may be formed by integrally molding the tube 10 and the distal end member 11.

The tube 10 is a long tubular member having flexibility and insulating properties. The tube 10 is made of, for example, resin. The distal end member 11 is fitted and attached to the inside of the tube 10. Furthermore, the distal end member 11 may be adhered to the tube 10 by an adhesive or the like.

The distal end member 11 is formed in a cylindrical shape. Furthermore, “cylindrical” includes not only a strict cylindrical shape but also a shape close to a cylindrical shape. The distal end member 11 is preferably made of an insulating material such as resin. A first through hole 12 is formed in the distal end member 11.

The first through hole 12 is a hole provided in the distal end member 11 and penetrating the distal end member 11 in the longitudinal direction A. The front end of the first through hole 12 communicates with a distal end opening 12a formed in a distal end surface 14 of the distal end member 11. The proximal end of the first through hole 12 communicates with an internal space 19 of the tube 10. The knife 2 is inserted through the first through hole 12.

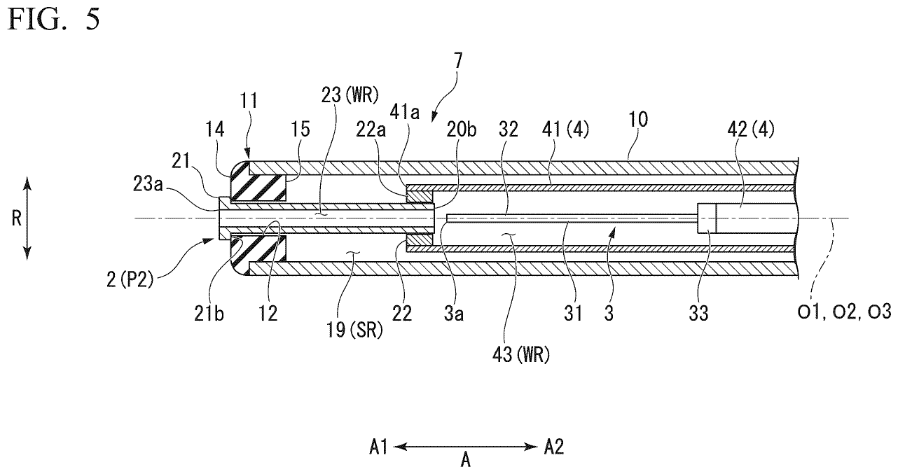

FIGS. 4 and 5 are cross-sectional views of the distal end portion of the treatment instrument 100.

The knife (electrode, knife electrode) 2 is a rod-shaped metal member. Furthermore, “round bar shape” includes not only a strictly round bar shape but also a shape close to a round bar shape. The knife 2 is made of a material such as stainless steel. The knife 2 is conductive and receives a high-frequency current. The knife 2 has a knife body 20, a flange 21, and a first connector 22.

The knife 2 is inserted through the first through hole 12 of the distal end member 11 of the outer sheath 1 along the longitudinal direction A, and is able to protrude and retract from the distal end opening 12a to the distal end side A1. Furthermore, the knife 2 may be fixed not to be advanceable and retractable while protruding from the distal end opening 12a to the distal end side A1.

A central axis O2 in the longitudinal direction A of the knife 2 preferably coincides with a central axis O1 in the longitudinal direction A of the outer sheath 1. Furthermore, “match” includes not only exact matches but also near matches.

The knife body 20 is a rod-shaped metal member. The outer diameter of the knife body 20 is smaller than the inner diameter of the first through hole 12. The knife body 20 is advanceable and retractable through the first through hole 12 in the longitudinal direction A.

The flange (distal end enlarged diameter portion) 21 is a disk-shaped conductive member provided at the distal end of the knife body 20. A length D2 of the flange 21 in a radial direction R perpendicular to the longitudinal direction A is longer than a length D1 of the knife body 20 in the radial direction R. A flat proximal end surface 21b is formed on the proximal end side A2 of the flange 21. Furthermore, the flange 21 is not limited to a disk shape, but may be triangular or hook-shaped.

The knife body 20 and the flange 21 have a first pipeline 23 extending along the longitudinal direction A. The first pipeline 23 communicates with a distal end opening 23a formed in the flange 21. The distal end opening 23a is an opening provided on the distal end side A1 of the flange 21

The first connector (butting member) 22 is a cylindrical-shaped metal member. Furthermore, “cylindrical” includes not only a strict cylindrical shape but also a shape close to a cylindrical shape. The first connector 22 connects the knife body 20 and an inner tube 41. Specifically, the outer peripheral surface of the knife body 20 and the inner peripheral surface of the first connector 22 are fixed to each other by welding or the like, and the outer peripheral surface of the first connector 22 and the inner peripheral surface of the inner tube 41 are fixed to each other by welding or the like. The first connector 22 cannot be inserted through the first through hole 12 in the longitudinal direction A. The first pipeline 23 communicates with an internal space 43 formed in the inner tube 41.

The inner tube 41 is attached to the proximal end of the knife body 20 through the first connector 22. A high-frequency current is supplied to the knife 2 via the inner tube 41 connected to the operation portion 5. When a high-frequency current is supplied from the inner tube 41 to the knife 2, the knife body 20 and the flange 21 function as active electrodes that output the high-frequency current to the biological tissue.

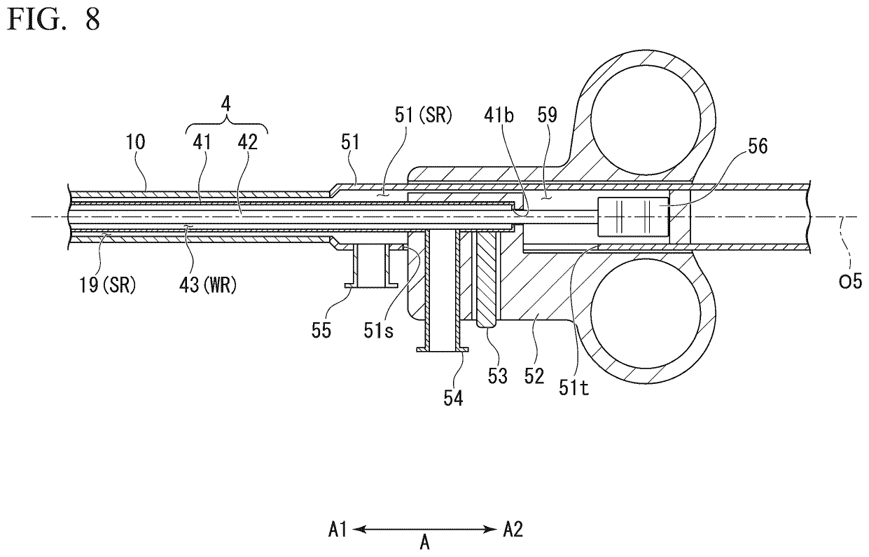

As shown in FIG. 4, when the knife 2 advances relative to the outer sheath 1, a distal end 22a of the first connector 22 or a distal end 41a of the inner tube 41 comes into contact with a proximal end surface 15 of the distal end member 11. The knife 2 is positioned at a first position P1 which is the position on the most distal end side A1 when the distal end 22a of the first connector 22 comes into contact with the proximal end surface 15 of the distal end member 11. Furthermore, the knife 2 may be positioned at the first position P1 when a slider 52 of the operation portion 5 to be described later comes into contact with a distal end stopper 51s (see FIG. 8).

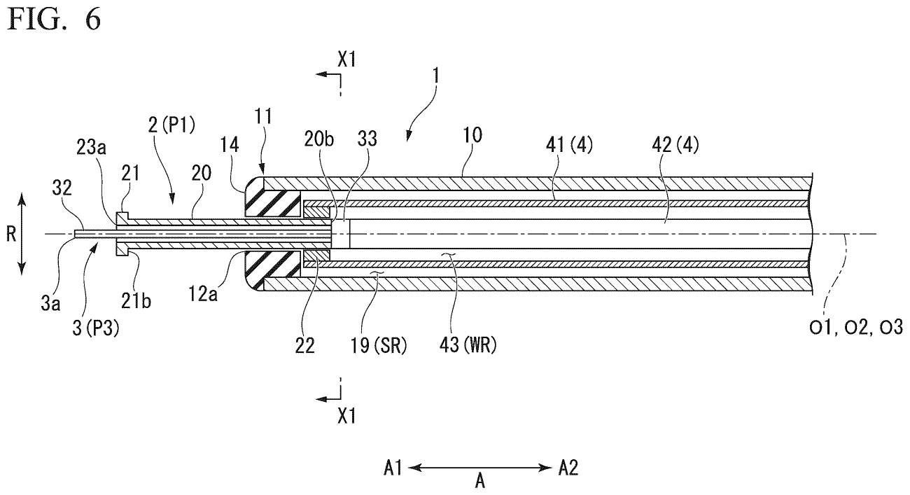

As shown in FIG. 5, when the knife 2 retracts relative to the outer sheath 1, the proximal end surface 21b of the flange 21 and the distal end surface 14 of the distal end member 11 come into contact with each other. When the proximal end surface 21b of the knife 2 and the distal end surface 14 of the distal end member 11 come into contact with each other, the knife 2 is positioned at a second position P2 which is the position on the most proximal end side A2. Furthermore, the knife 2 may be positioned at the second position P2 when the slider 52 of the operation portion 5 to be described later comes into contact with a proximal end stopper 51t (see FIG. 8). FIG. 6 is a cross-sectional view of the distal end portion of the treatment instrument 100 when the rod 3 is in a third position P3.

The rod (solid needle) 3 is a rod-shaped member, and is preferably made of a non-conductive material such as a resin material or a ceramic material not to be electrically conductive against the intention of the surgeon, but may be made of a metal material. The rod 3 has a main body portion 31, a distal end portion 32, and a second connector 33.

The rod 3 is inserted through the first pipeline 23 of the knife 2 along the longitudinal direction A, and is capable of protruding and retracting from the distal end opening 23a toward the distal end side A1.

The main body portion 31 and the distal end portion 32 are long round bar-shaped members. Furthermore, “round bar shape” includes not only a strictly round bar shape but also a shape close to a round bar shape. The outer diameter of the main body portion 31 and the distal end portion 32 is smaller than the inner diameter of the first pipeline 23. The main body portion 31 and the distal end portion 32 can advance and retract the first pipeline 23 in the longitudinal direction A. The distal end portion 32 may be formed so that the distal end side A1 is tapered.

The second connector 33 connects the rod 3 and an operation wire 42. Specifically, the proximal end of the rod 3 and the distal end of the operation wire 42 are fixed by welding or the like. The second connector 33 cannot be inserted through the first pipeline 23 in the longitudinal direction A.

As shown in FIG. 6, when the rod 3 advances relative to the outer sheath 1, the second connector 33 of the rod 3 comes into contact a proximal end 20b of the knife body 20. The distal end portion 32 protrudes from the distal end opening 23a to the distal end side A1. When the second connector 33 of the rod 3 comes into contact the proximal end 20b of the knife body 20, the rod 3 is positioned at the third position P3 which is the position on the most distal end side A1.

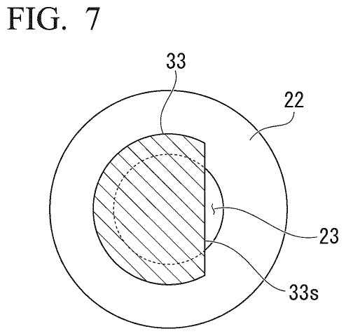

FIG. 7 is a cross-sectional view taken along line X1-X1 shown in FIG. 6.

The second connector 33 has a cross section perpendicular to the longitudinal axis direction A that is not circular, but has a shape in which a part of the circular shape is cut out (for example, a D-cut shape). That is, at least a part of the traverse cross section of the second connector 33 at the distal end portion is formed by an outer surface 33s extending in a direction perpendicular to the longitudinal axis of the rod 3. The number of outer surfaces 33s is not limited to one, but may be multiple. Further, the shape with a part of a circular shape cut out may be a shape having a concave outer surface. Furthermore, the outer surface 33s may be formed over the entire length of the second connector 33 from the distal end to the proximal end. With this configuration, even when the second connector 33 comes into contact with the proximal end of the knife body 20 as shown in FIG. 6, the internal space 43 of the inner tube 41 communicates with the first pipeline 23. Furthermore, the shape of the second connector 33 is not limited thereto as long as it is a shape that can form a flow passage communicating between the internal space 43 and the first pipeline 23 when the second connector 33 comes into contact with the proximal end of the knife body 20. For example, the distal end portion of the second connector 33 is not limited to a circular shape with a part cut out, and may be a rectangular or elliptical shape. FIG. 8 is a cross-sectional view of the operation member 4 and the operation portion 5.

The operation member 4 has an inner tube 41 which is inserted through the internal space (pipeline, lumen) 19 of the outer sheath 1 and an operation wire 42 which is inserted through the internal space 43 of the inner tube 41.

The inner tube (first operation member) 41 is a conductive wire that operates the knife 2. The distal end of the inner tube 41 is connected to the knife 2 through the first connector 22. The proximal end of the inner tube 41 is connected to the slider 52 of the operation portion 5. Furthermore, the inner tube 41 may be in any other form as long as the inner tube is a hollow conductive tube, for example, a hollow metal coil.

The operation wire (second operation member) 42 is a wire that operates the rod 3. The distal end of the operation wire 42 is connected to the rod 3 through the second connector 33. The proximal end of the operation wire 42 passes through a through hole 41b provided in the proximal end of the inner tube 41 and is connected to a lever 56 of the operation portion 5. Furthermore, the operation wire 42 may be in any other form as long as the operation wire is a member capable of advancing and retracting the rod 3.

Furthermore, the rod 3 and the operation wire 42 may be integrally formed with each other. For example, the rod 3 and the operation wire 42 may be a single wire or a stranded wire, and may be processed so that the distal end portion has a smaller diameter than the proximal end portion.

The operation portion (handle) 5 has, as shown in FIG. 2, a handle body 51, a slider 52, a power supply plug 53, a liquid delivery port 54, a suction port 55, a lever 56, and an elastic body 57.

The distal end portion of the handle body 51 is connected to the proximal end 1b of the outer sheath 1. The handle body 51 has an internal space 59 through which the inner tube 41 is insertable. The inner tube 41 passes through the internal space 19 of the tube 10 and the internal space 59 of the handle body 51 and extends to the slider 52. The operation wire 42 passes through the internal space 43 of the inner tube 41 and the internal space 59 of the handle body 51 and extends to the lever 56.

The slider (second slider) 52 is attached to the handle body 51 to be movable along the longitudinal direction A. The proximal end of the inner tube 41 is attached to the slider 52. When the surgeon advances and retracts the slider 52 relative to the handle body 51, the inner tube 41 and the knife 2 advance and retract.

The power supply plug 53 is fixed to the slider 52. The power supply plug 53 can be connected to a high-frequency power supply device (not shown) and is connected to the proximal end portion of the inner tube 41. The power supply plug 53 can supply a high-frequency current supplied from the high-frequency power supply device to the knife 2 via the inner tube 41. Furthermore, the power supply plug 53 may be fixed to the handle body 51 instead of the slider 52.

The liquid delivery port 54 is provided in the slider 52. The liquid delivery port 54 is connected to the proximal end portion of the inner tube 41 and communicates with the internal space 43. The liquid supplied from the liquid delivery port 54 passes through a water delivery flow passage WR (internal space 43, first pipeline 23) and is discharged from the distal end opening 23a. Furthermore, the liquid delivery port 54 may be provided in the handle body 51 instead of the slider 52.

The suction port 55 is provided in the handle body 51. The suction port 55 is connected to the distal end portion of the handle body 51 and communicates with the internal space 59. The suction port 55 is connectable to a pump device (not shown). By suctioning from the suction port 55, the liquid sucked from the distal end opening 12a passes through the suction flow passage SR (internal space 19, internal space 59) and is discharged outside the body.

The lever (first slider) 56 is attached to the slider 52 to be movable along the longitudinal direction A. The proximal end of the operation wire 42 is attached to the lever 56. When the surgeon advances and retracts the lever 56 relative to the slider 52, the operation wire 42 and the rod 3 advance and retract.

The elastic body 57 is located between the handle body 51 and the lever 56, and connects the handle body 51 and the lever 56. The distal end of the elastic body 57 is attached to the handle body 51, and the proximal end of the elastic body 57 is attached to the lever 56. The elastic body 57 is connected to the operation wire 42 through the lever 56. The elastic body 57 biases the lever 56 toward the proximal end side A2 so that a distal end 3a of the rod 3 is accommodated in the internal space 43 of the inner tube 41. That is, the distal end 3a of the rod 3 is located inside the inner tube 41 while the elastic body 57 returns to an original shape, and when the rod 3 advances against the elastic force of the elastic body 57, the rod 3 is biased by the elastic body 57 toward the proximal end side A2. Furthermore, the distal end 3a of the rod 3 is located on the proximal end side A2 in relation to the distal end of the outer sheath 1 while the elastic body 57 returns to an original shape. The elastic body 57 may bias the distal end of the rod 3 so that the distal end of the rod 3 is accommodated in more proximal side than the distal end of the knife 2. Specifically, the elastic body 57 may be a spring, or may be rubber instead of a spring.

Furthermore, the lever 56 may be composed of two or more parts. For example, the lever 56 may be composed of a first member that is connected to the operation wire 42 and the elastic body 57, and a second member that is operated by the surgeon in a contact state.

### Operation of Treatment Instrument 100

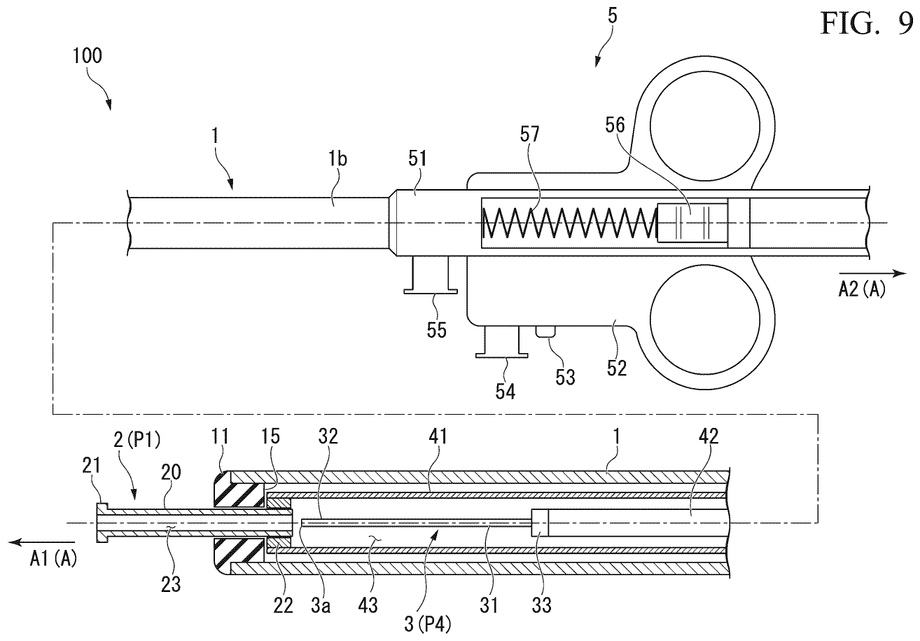

Next, the operation of the treatment instrument 100 will be described. FIGS. 9 to 12 are diagrams illustrating the operation of the treatment instrument 100.

As shown in FIG. 9, the surgeon advances the slider 52 relative to the handle body 51 so that the knife 2 advances relative to the outer sheath 1. The knife 2 is positioned at the first position P1. Since the elastic body 57 biases the lever 56 toward the proximal end side A2, the distal end 3a of the rod 3 remains in a position (fourth position P4) to be accommodated in the internal space 43 of the inner tube 41. At this time, the distal end 3a of the rod 3 is disposed on the proximal end side A2 in relation to the distal end of the outer sheath 1.

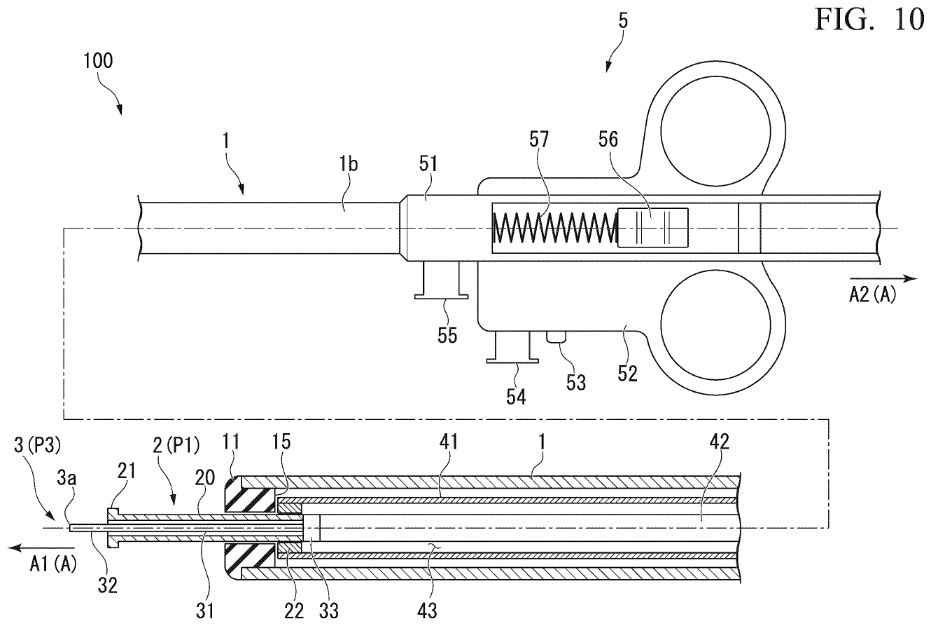

As shown in FIG. 10, the surgeon advances the lever 56 relative to the slider 52 so that the rod 3 advances relative to the knife 2. The rod 3 is positioned at the third position P3, and the distal end portion 32 protrudes from the distal end opening 23a to the distal end side A1. When the surgeon releases the lever 56, the lever 56 moves to the proximal end side A2 due to the elastic force of the elastic body 57. As a result, as shown in FIG. 9, the distal end 3a of the rod 3 returns to the fourth position P4 to be accommodated in the internal space 43 of the inner tube 41.

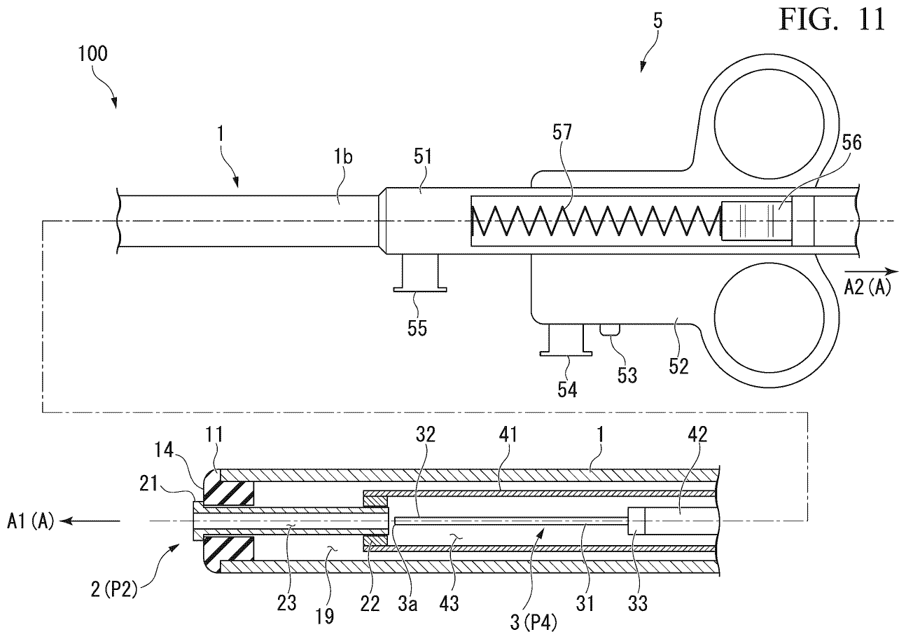

As shown in FIG. 11, the surgeon retracts the slider 52 relative to the handle body 51 so that the knife 2 retracts relative to the outer sheath 1. The knife 2 is positioned at the second position P2. Since the elastic body 57 biases the lever 56 toward the proximal end side A2, the distal end 3a of the rod 3 moves to the fourth position P4 to be accommodated in the internal space 43 of the inner tube 41. At this time, the distal end 3a of the rod 3 is disposed on the proximal end side A2 in relation to the distal end of the outer sheath 1.

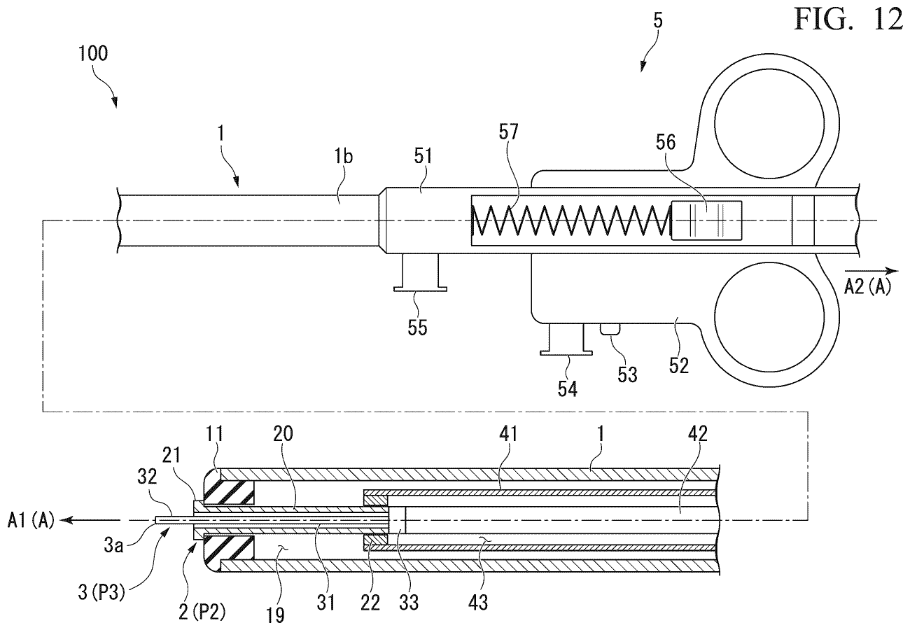

As shown in FIG. 12, the surgeon advances the lever 56 relative to the slider 52 so that the rod 3 advances relative to the knife 2. The rod 3 is positioned at the third position P3, and the distal end portion 32 protrudes from the distal end opening 23a to the distal end side A1. When the surgeon releases the lever 56, the lever 56 moves to the proximal end side A2 due to the elastic force of the elastic body 57. As a result, as shown in FIG. 11, the distal end 3a of the rod 3 moves to the fourth position P4 to be accommodated in the internal space 43 of the inner tube 41.

### Method of Using Endoscopic Treatment System 300

Next, a procedure using the endoscopic treatment system 300 of this embodiment (a method of using the endoscopic treatment system 300) will be described. Specifically, local injection treatment and incision and dissection treatment of a lesion in endoscopic treatment such as endoscopic submucosal dissection (ESD) will be described.

As a preparatory step, the surgeon identifies the lesion by a known method. Specifically, the surgeon inserts the insertion portion 202 of the endoscope 200 into the digestive tract (for example, the esophagus, stomach, duodenum, or large intestine) and identifies the lesion while observing images obtained by the imaging unit 203 of the endoscope

### Inserting Step

The surgeon inserts the treatment instrument 100 into the channel 206 so that the distal end 1a of the outer sheath 1 protrudes from the distal end opening portion 206a of the insertion portion 202.

### Marking Step

As shown in FIG. 11, the surgeon retracts the slider 52 relative to the handle body 51 so that the knife 2 is disposed at the second position P2. The flange 21 protruding from the distal end surface 14 of the distal end member 11 is used to cauterize and mark the biological tissue surrounding the lesion.

### Puncture Step

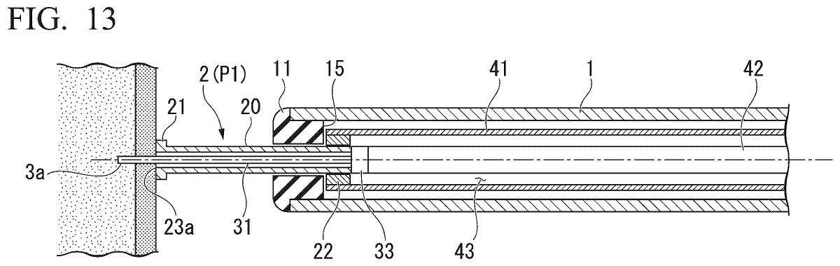

FIG. 13 is a diagram showing the rod 3 puncturing the tissue.

As shown in FIG. 10, the surgeon advances the slider 52 relative to the handle body 51 so that the knife 2 is disposed at the first position P1. Further, the surgeon advances the lever 56 relative to the slider 52 so that the rod 3 is disposed at the third position P3. The surgeon uses the rod 3 to puncture and penetrate the site at the lesion where a liquid for local injection (local injection liquid) is to be injected.

### Local Injection Step

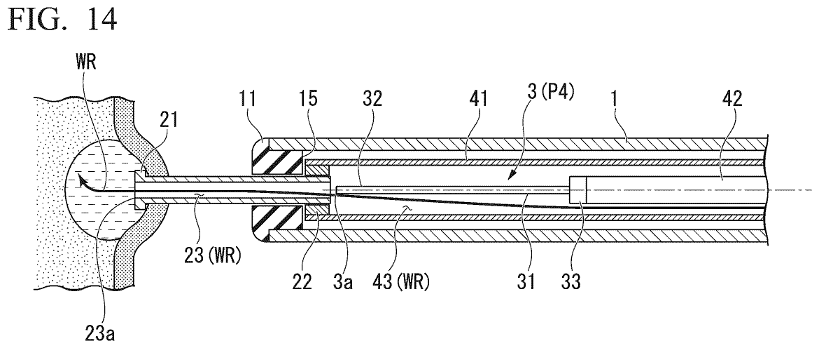

FIG. 14 is a diagram showing the knife 2 that delivers liquid into tissue.

As shown in FIG. 9 or 11, when the surgeon releases the lever 56, the rod 3 retracts to the fourth position P4. Accordingly, the water delivery flow passage WR (internal space 43, first pipeline 23) is widely ensured. The surgeon inserts the distal end (including the distal end opening 23a) of the knife 2 into the submucosal layer through the hole formed by puncturing with the rod 3 or delivers liquid (local injection liquid) from the liquid delivery port 54 into the tissue while being in contact with the tissue surface. The liquid (local injection liquid) is discharged from the distal end opening 23a and injected into the tissue.

### Incision and Dissection Step

Next, the surgeon performs the incision and dissection procedure. As shown in FIG. 9, the surgeon advances the knife 2 and moves the flange 21 to incise the mucous membrane of the lesion while a high-frequency current is being applied thereto. Further, the surgeon advances the knife 2, lifts the mucous membrane of the incised lesion to expose the submucosal layer, and dissects the submucosal layer of the incised lesion with high-frequency current being applied.

The surgeon continues the above-described operations (treatments) as necessary, finally excises the lesion, and completes the ESD procedure.

According to the treatment instrument 100 of this embodiment, the knife 2 and the rod 3 can be easily operated according to the treatment situation, such as incision and dissection treatment and local injection treatment. The surgeon does not need to operate the lever 56 retracted, and when the surgeon releases the lever 56, the rod 3 is automatically stored in the internal space 43 of the inner tube 41, and hence the procedure is simplified. Further, it is possible to prevent the surgeon from unintentionally operating the knife 2 with the rod 3 protruding.

Although the first embodiment of the present disclosure has been described above in detail with reference to the drawings, the specific configuration is not limited to this embodiment, and design changes and the like are also included within the scope of the present disclosure. Further, the components shown in the embodiments and modified examples can be appropriately configured in combination.

### Modified Example 1

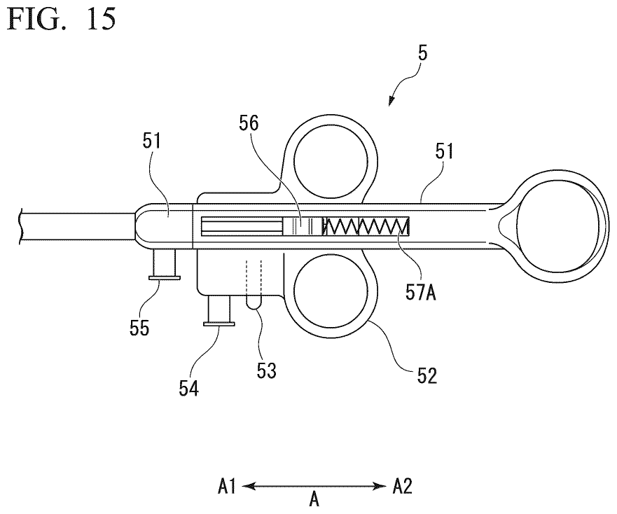

FIG. 15 is a diagram showing an elastic body 57A which is a modified example of the elastic body 57. The distal end of the elastic body 57A is attached to the lever 56, and the proximal end of the elastic body 57A is attached to the handle body 51. When the lever 56 advances relative to the handle body 51, a restoring force (elastic force) acts on the elastic body 57A as the elastic body 57A deforms, and the lever 56 moves toward the proximal end side A2. As in the elastic body 57, the elastic body 57A biases the lever 56 toward the proximal end side A2 so that the internal space 43 of the inner tube 41 is accommodated in the first pipeline 23 of the knife 2. That is, as in the elastic body 57, the distal end 3a of the rod 3 is located inside the inner tube 41 while the elastic body 57A returns to an original shape, and when the rod 3 advances against the elastic force of the elastic body 57A, the rod 3 is biased by the elastic body 57 toward the proximal end side A2. Furthermore, the distal end 3a of the rod 3 is located on the proximal end side A2 in relation to the distal end of the outer sheath 1 while the elastic body 57A returns to an original shape. Specifically, the elastic body 57A may be a spring, or may be rubber instead of a spring.

### Modified Example 2

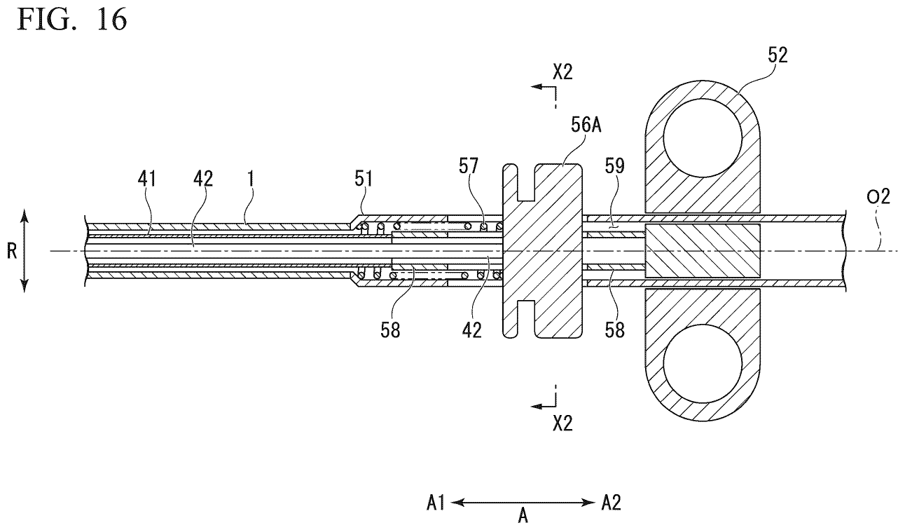

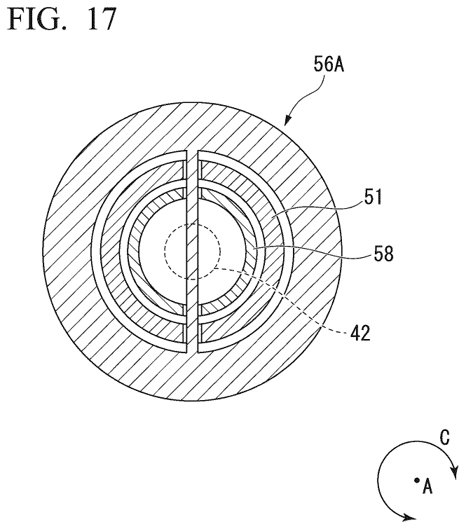

FIG. 16 is a diagram showing a lever 56A which is a modified example of the lever 56. FIG. 17 is a cross-sectional view taken along line X2-X2 shown in FIG. 16. The lever 56A is attached to the outer peripheral portion of the handle body 51 instead of the slider 52, and is attached to the handle body 51 to be movable along the longitudinal direction A. A hollow pipe 58 is provided in the internal space 59 of the handle body 51. The pipe 58 connects the inner tube 41 and the slider 52. The operation wire 42 is inserted through the pipe 58, and is connected to the lever 56A. The elastic body 57 is inserted through the pipe 58, and connects the handle body 51 and the lever 56A. Since the lever 56A is disposed at a position separated from the slider 52, the surgeon can operate the lever 56A independently of the slider 52. That is, the lever 56A does not move even when the slider 52 is moved along the longitudinal direction A, and the slider 52 does not move even when the lever 56A is moved along the longitudinal direction A.

### Modified Example 3

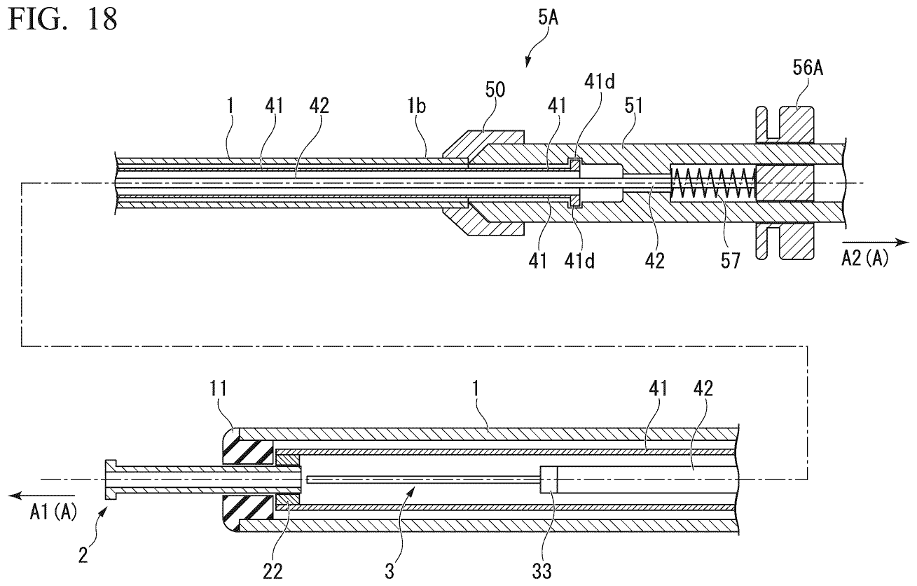

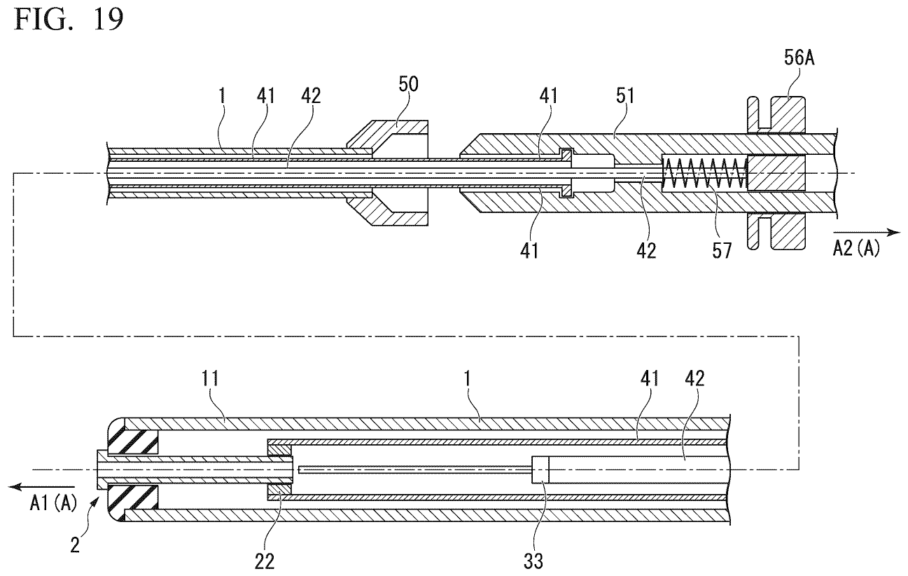

FIGS. 18 and 19 are diagrams showing an operation portion 5A which is a modified example of the operation portion 5. The operation portion 5A has the sheath slider 50, the handle body 51, the power supply plug 53, the liquid delivery port 54, the suction port 55, the lever 56A, and the elastic body 57. Furthermore, the power supply plug 53, the liquid delivery port 54, and the suction port 55 are not shown. The sheath slider 50 is connected to the proximal end 1b of the outer sheath 1, and is advanceable and retractable relative to the handle body 51. The proximal end 41d of the inner tube 41 is attached to the handle body 51 not to be advanceable and retractable. The surgeon advances and retracts the sheath slider 50 relative to the handle body 51 so that the outer sheath 1 can advance and retract relative to the knife 2. Furthermore, the inner tube 41 may be advanceable and retractable together with the outer sheath 1.

### Modified Example 4

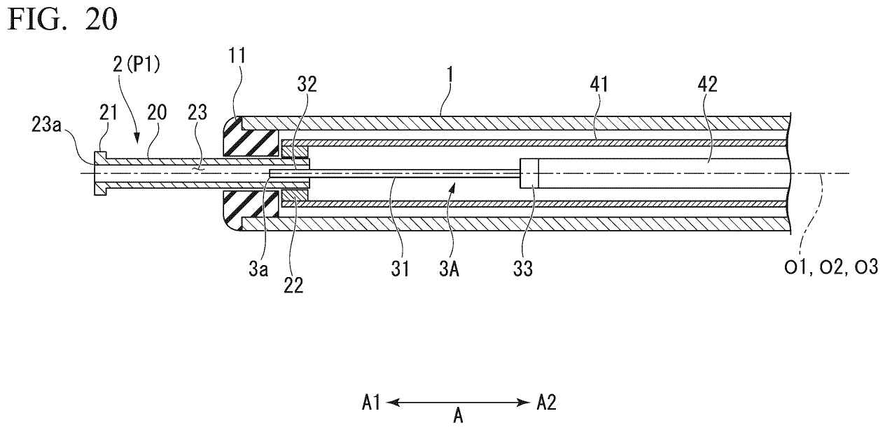

FIG. 20 is a diagram showing a rod 3A which is a modified example of the rod 3. When the surgeon releases the lever 56, the rod 3A moves to a position in which the distal end 3a is accommodated in the first pipeline 23 of the knife 2. In this case, the distal end 3a of the rod 3A may be located between the distal end and the proximal end of the distal end member 11 in the longitudinal axis direction A.

### Modified Example 5

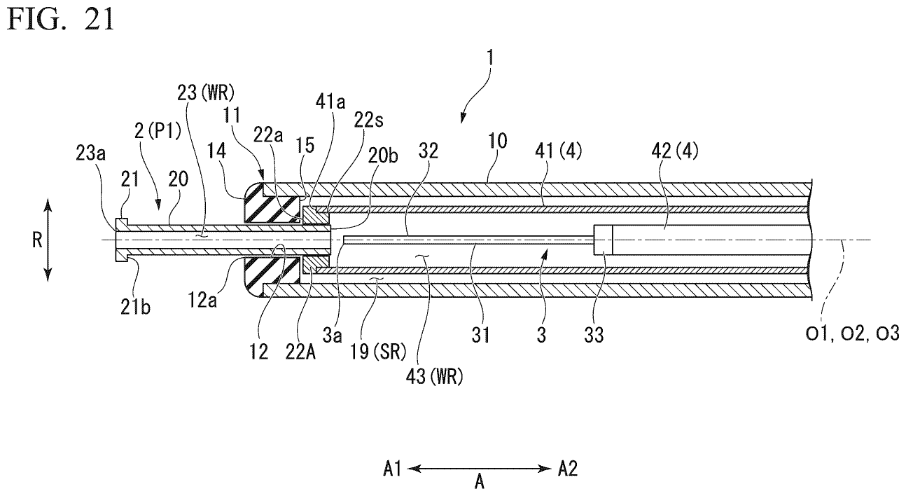

FIG. 21 is a diagram showing a first connector 22A which is a modified example of the first connector 22. In the above-described embodiment, the distal end 41a of the inner tube 41 attached to the first connector 22 is exposed to the distal end 22a of the first connector 22, and can come into contact with the proximal end surface 15 of the distal end member 11. On the other hand, the first connector 22A shown in FIG. 21 has a step 22s on the outer surface that engages with the distal end 41a of the inner tube 41. The distal end 41a of the inner tube 41 is not exposed to the distal end 22a of the first connector 22. The distal end 22a of the first connector 22A is reliably positioned by coming into contact with the proximal end surface 15 of the distal end member 11. The distal end 41a of the inner tube 41 can be easily connected to the step 22s of the first connector 22A, and the assembly is facilitated.

### Modified Example 6

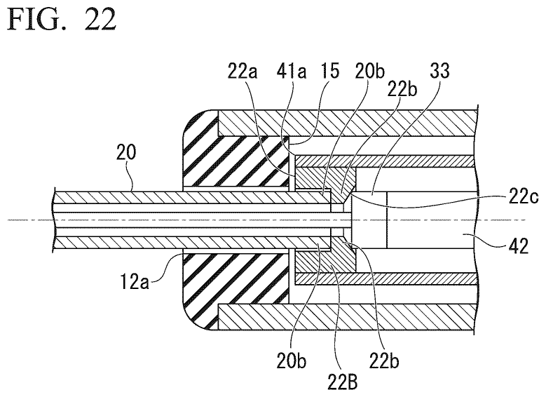

FIG. 22 is a diagram showing a first connector 22B which is a modified example of the first connector 22. The first connector 22B has a protrusion 22b extending radially inward on the inner surface at the proximal end side, and the knife body 20 is fixed by welding or the like while the knife body 20 comes into contact with the protrusion 22b. The protrusion 22b of the first connector 22B is located on the proximal end side A2 in relation to the proximal end 20b of the knife body 20. An inclined surface (tapered surface) 22c is formed on the proximal end side A2 of the protrusion 22b to come into contact with the second connector 33. The inclined surface 22c is formed in a tapered shape that decreases in diameter from the proximal end side A2 toward the distal end side A1. Therefore, when the lever 56 advances relative to the slider 52 while the distal end 3a of the rod 3 is located inside the internal space 43, the rod 3 can be smoothly inserted into the knife body 20 without the distal end 3a of the rod 3 getting caught on the first connector 22B. When the rod 3 advances relative to the inner tube 41 (knife body 20), the second connector 33 of the rod 3 and the inclined surface 22c of the first connector 22B come into contact with each other. Since the second connector 33 of the rod 3 and the first connector 22B come into contact with each other, the rod 3 is positioned at the third position P3 which is the position on the most distal end side A1. Furthermore, the protrusion 22b may have a reduced diameter shape. Even when the second connector 33 and the first connector 22B come into contact with each other, the internal space 43 of the inner tube 41 communicates with the first pipeline 23. The shape of the second connector 33 is not limited as long as it is a shape that can form a flow passage that communicates between the internal space 43 and the first pipeline 23 when the first connector 22B and the second connector 33 come into contact with each other. Further, if the second connector 33 does not have these flow passage forming shapes, a slit that can become a flow passage when the first connector 22B and the second connector 33 come into contact with each other may be formed on the tapered surface 22c.

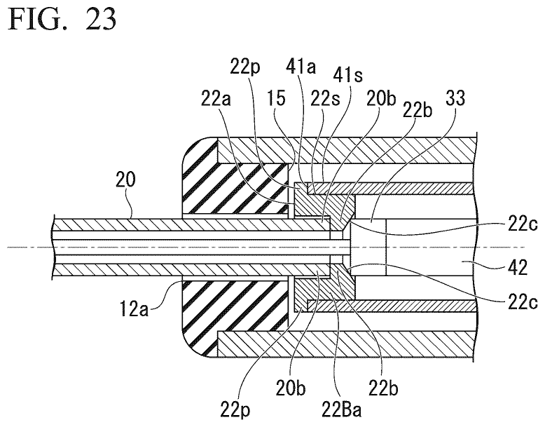

FIG. 23 is a diagram showing a first connector 22Ba which is a modified example of the first connector 22B. The first connector 22Ba has a protrusion 22p extending radially outward on the outer surface of the distal end side A1 of the first connector 22Ba, and the outer surface of the proximal end side of the first connector 22Ba is covered with a distal end portion 41s of the inner tube 41. The distal end 41a of the inner tube 41 is fixed to the protrusion 22p by welding or the like in a contact state. Therefore, the distal end 41a of the inner tube 41 is not exposed to the distal end 22a of the first connector 22Ba. According to this configuration, the distal end 22a of the first connector 22Ba is reliably positioned by coming into contact with the proximal end surface 15 of the distal end member 11. The distal end 41a of the inner tube 41 can be easily connected to the step 22s of the first connector 22Ba, and the assembly is facilitated. Even when the second connector 33 and the first connector 22Ba come into contact with each other, the internal space 43 of the inner tube 41 communicates with the first pipeline 23. The shape of the second connector 33 is not limited as long as it is a shape that can form a flow passage that communicates between the internal space 43 and the first pipeline 23 when the first connector 22Ba and the second connector 33 come into contact with each other. Further, if the second connector 33 does not have these flow passage forming shapes, a slit that can become a flow passage when the first connector 22Ba and the second connector 33 come into contact with each other may be formed on the tapered surface 22c.

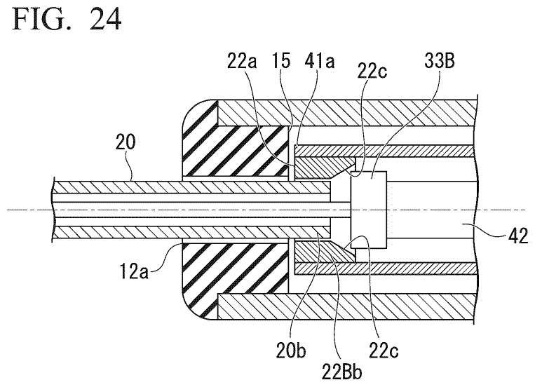

FIG. 24 is a diagram showing a first connector 22Bb which is a modified example of the first connector 22B. A second connector 33B is a connector having a larger outer diameter than the second connector 33. The inclined surface 22c of the first connector 22Bb extends outward in the radial direction R compared to the inclined surface 22c of the first connector 22B. The inclined surface 22c of the first connector 22Bb has a length (outer diameter) in the radial direction R that is longer than the outer diameter of the knife body 20, and can be in sufficient contact with the second connector 33B. That is, the second connector 33B comes into contact with the inclined surface 22c of the first connector 22Bb, and does not come into contact with the knife body 20. Even in this configuration, the distal end 22a of the first connector 22Bb is reliably positioned by coming into contact with the proximal end surface 15 of the distal end member 11. Even in this configuration, the distal end 22a of the first connector 22Bb contacts the proximal end surface 15 of the distal end member 11 and is reliably positioned. Even when the second connector 33B and the first connector 22Bb come into contact with each other, the internal space 43 of the inner tube 41 communicates with the first pipeline 23. The shape of the second connector 33B is not limited as long as it is a shape that can form a flow passage that communicates between the internal space 43 and the first pipeline 23 when the first connector 22Bb and the second connector 33B come into contact with each other. Further, if the second connector 33B does not have these flow passage forming shapes, a slit that can become a flow passage when the first connector 22Bb and the second connector 33B come into contact with each other may be formed on the tapered surface 22c.

### Modified Example 7

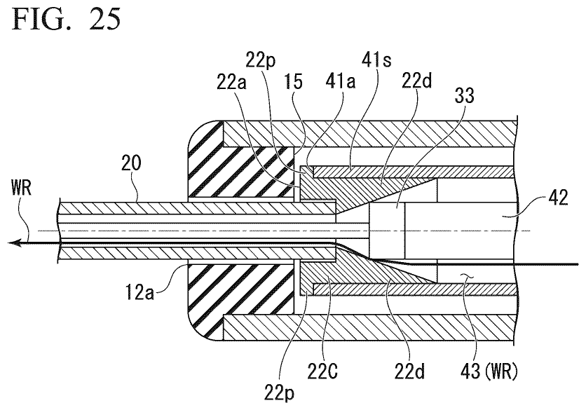

FIG. 25 is a diagram showing a first connector 22C which is a modified example of the first connector 22. The first connector 22C has an inclined pipeline 22d which is located on the proximal end side A2 in relation to the proximal end 20b of the knife body 20. The inclined pipeline 22d is a pipeline through which the rod 3 is inserted. The inner peripheral surface of the inclined pipeline 22d is formed in a tapered shape that decreases in diameter from the proximal end side A2 toward the distal end side A1. Therefore, when the lever 56 advances relative to the slider 52 while the distal end 3a of the rod 3 is located inside the internal space 43, the rod 3 can be smoothly inserted into the knife body 20 without the distal end 3a of the rod 3 getting caught on the first connector 22C. When the rod 3 advances relative to the inner tube 41 (knife body 20), the second connector 33 of the rod 3 and the inner peripheral surface of the inclined pipeline 22d of the first connector 22C come into contact with each other. When the second connector 33 comes into contact with the inner peripheral surface of the inclined pipeline 22d, the second connector 33 is located inside the inclined pipeline 22d. Further, at least a part of the traverse cross section of the second connector 33 at the distal end portion is the outer surface 33s extending in a direction perpendicular to the longitudinal axis of the rod 3 as shown in FIG. 7. The shape of the second connector 33 is not limited as long as it is a shape that can form a flow passage that communicates between the internal space 43 and the first pipeline 23 when the first connector 22C and the second connector 33 come into contact with each other. Further, when the second connector 33 does not have these flow passage forming shapes, a slit that can become a flow passage when the first connector 22C and the second connector 33 come into contact with each other may be formed on the inner peripheral surface of the inclined pipeline 22d.

### Modified Example 8

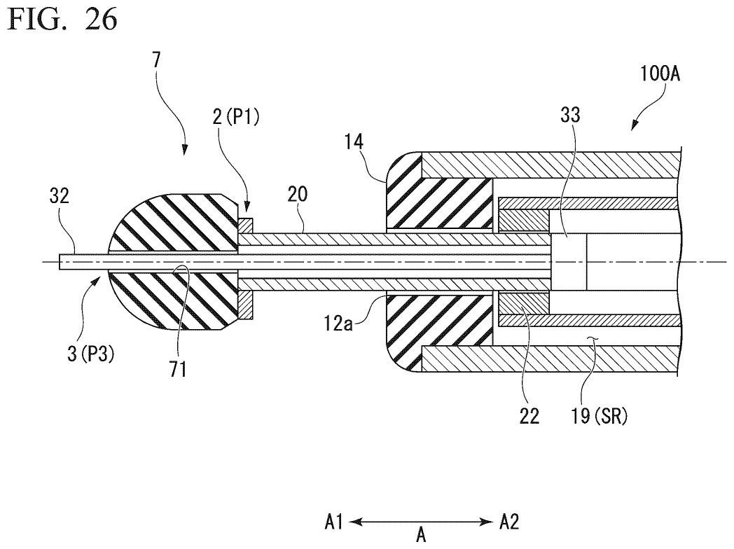

FIG. 26 is a diagram showing a distal end portion of a treatment instrument 100A which is a modified example of the treatment instrument 100. The treatment instrument 100A further includes an insulating member 7. The insulating member 7 is a non-conductive member such as a resin or ceramic material. The insulating member 7 is provided at the distal end of the knife body 20. The insulating member 7 has a through hole 71 that penetrates in the longitudinal axis direction A. The through hole 71 communicates with the first pipeline 23. The distal end portion 32 of the rod 3 can pass through the first pipeline 23 and the through hole 71 and protrude from the through hole 71 to the distal end side A1.

### Second Embodiment

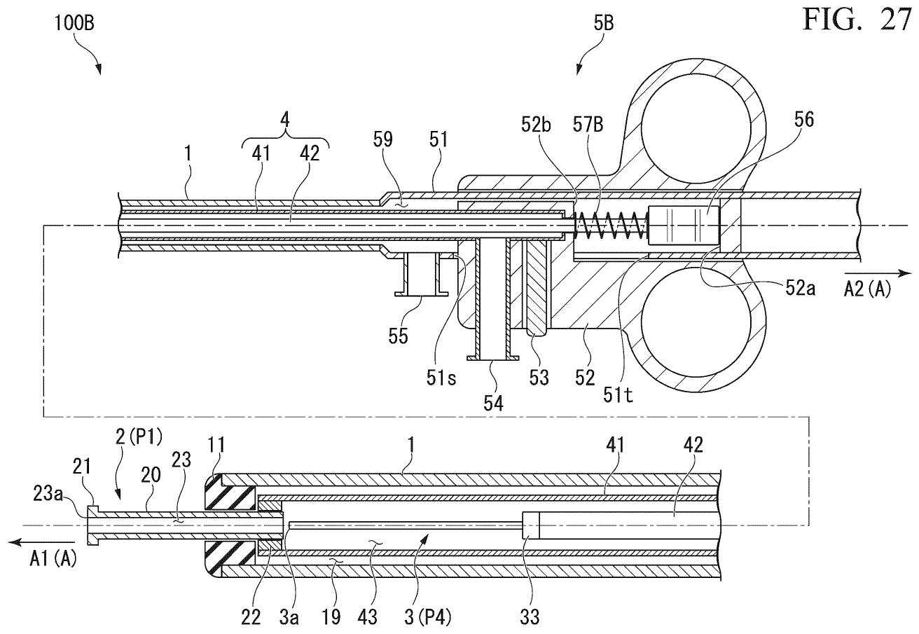

A treatment instrument 100B according to a second embodiment of the present disclosure will be described with reference to FIGS. 27 to 30. In the following description, the same components as those already described will be denoted by the same reference numerals and the description thereof will be omitted.

FIGS. 27 to 30 are diagrams illustrating the operation of the treatment instrument 100B.

The treatment instrument (endoscopic treatment instrument) 100B includes the outer sheath 1, the knife 2, the rod 3, the operation member 4, and an operation portion 5B

The operation portion (handle) 5B has the handle body 51, the slider 52, the power supply plug 53, the liquid delivery port 54, the suction port 55, the lever 56, and an elastic body 57B.

The elastic body 57B is located between the slider 52 and the lever 56, and connects the slider 52 and the lever 56. The distal end of the elastic body 57B is attached to the slider 52, and the proximal end of the elastic body 57B is attached to the lever 56. The elastic body 57B is connected to the operation wire 42 through the lever 56. The elastic body 57B biases the lever 56 toward the proximal end side A2 so that the distal end 3a of the rod 3 is accommodated in the internal space 43 of the inner tube 41. That is, as in the elastic body 57, the distal end 3a of the rod 3 is located inside the inner tube 41 while the elastic body 57B returns to an original shape, and when the rod 3 advances against the elastic force of the elastic body 57B, the rod 3 is biased by the elastic body 57B toward the proximal end side A2. Furthermore, the distal end 3a of the rod 3 is located on the proximal end side A2 in relation to the distal end of the outer sheath 1 while the elastic body 57B returns to an original shape. Specifically, the elastic body 57B may be a spring, or may be rubber instead of a spring.

As shown in FIG. 27, the surgeon advances the slider 52 relative to the handle body 51 so that the knife 2 advances relative to the outer sheath 1. The knife 2 is positioned at the first position P1. Since the elastic body 57B biases the lever 56 toward the proximal end side A2, the distal end 3a of the rod 3 remains at the fourth position P4 to be accommodated in the internal space 43 of the inner tube 41. At this time, the distal end 3a of the rod 3 is disposed on the proximal end side A2 in relation to the distal end of the outer sheath 1.

When the slider 52 advances relative to the handle body 51, the lever 56 engages with a first receiving surface 52a formed on the proximal end of the slider 52 and advances together with the slider 52 while being pressed by the first receiving surface 52a. Therefore, the relative positional relationship between the knife 2 and the rod 3 in the longitudinal axis direction A is maintained.

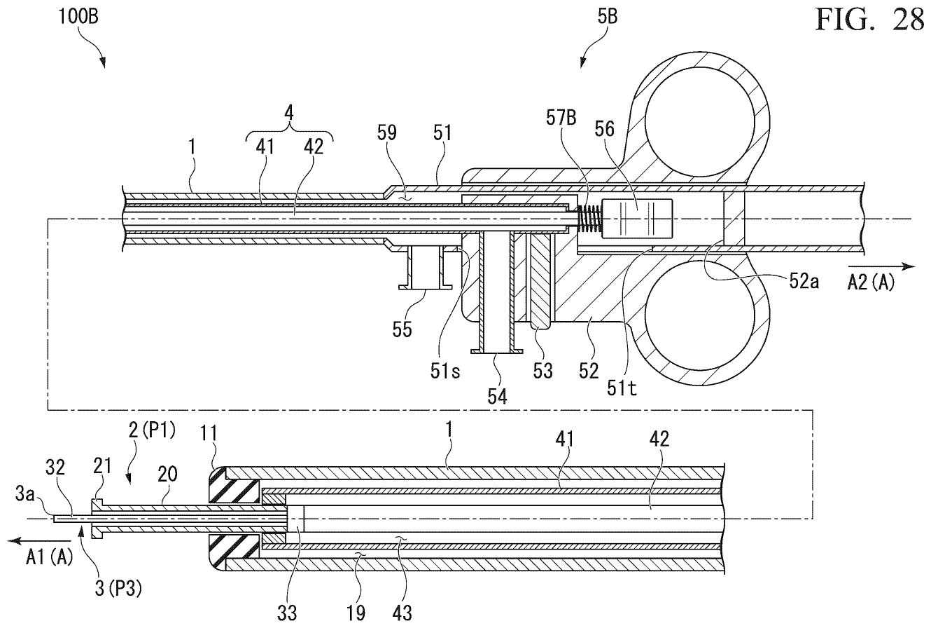

As shown in FIG. 28, the surgeon advances the lever 56 relative to the slider 52 so that the rod 3 advances relative to the knife 2. The rod 3 is positioned at the third position P3, and the distal end portion 32 protrudes from the distal end opening 23a to the distal end side A1. When the surgeon releases the lever 56, the lever 56 moves to the proximal end side A2 due to the elastic force of the elastic body 57B. As a result, as shown in FIG. 27, the distal end 3a of the rod 3 moves to the fourth position P4 to be accommodated in the internal space 43 of the inner tube 41.

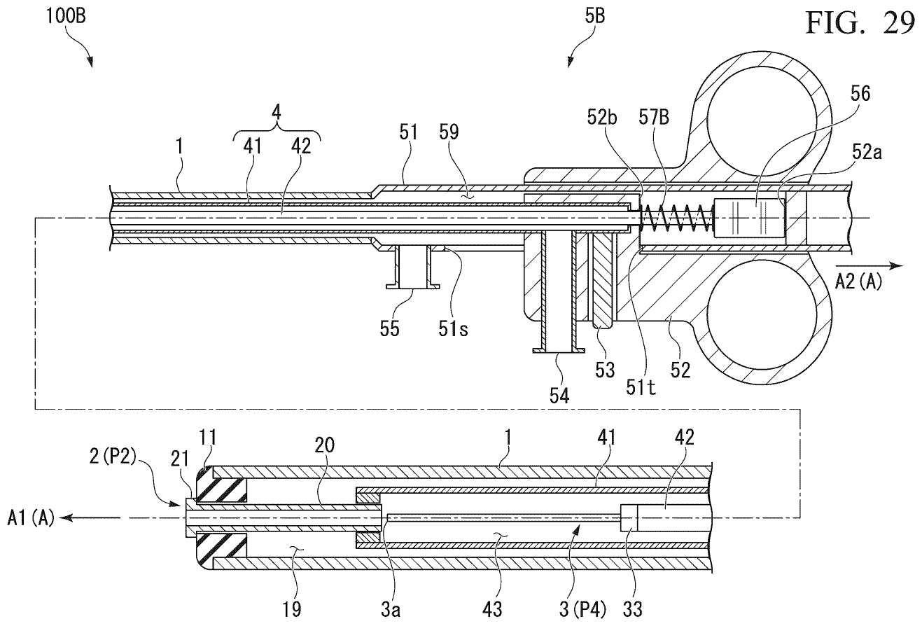

As shown in FIG. 29, the surgeon retracts the slider 52 relative to the handle body 51 so that the knife 2 retracts relative to the outer sheath 1. The knife 2 is positioned at the second position P2. Since the elastic body 57B biases the lever 56 toward the proximal end side A2, the distal end 3a of the rod 3 moves to the fourth position P4 to be accommodated in the internal space 43 of the inner tube 41. At this time, the distal end 3a of the rod 3 is disposed on the proximal end side A2 in relation to the distal end of the outer sheath 1.

When the slider 52 retracts relative to the handle body 51, the lever 56 retracts together with the slider 52 while being pressed by the elastic body 57B which is pressed by a second receiving surface 52b formed on the slider 52. Therefore, the relative positional relationship between the knife 2 and the rod 3 in the longitudinal axis direction A is maintained.

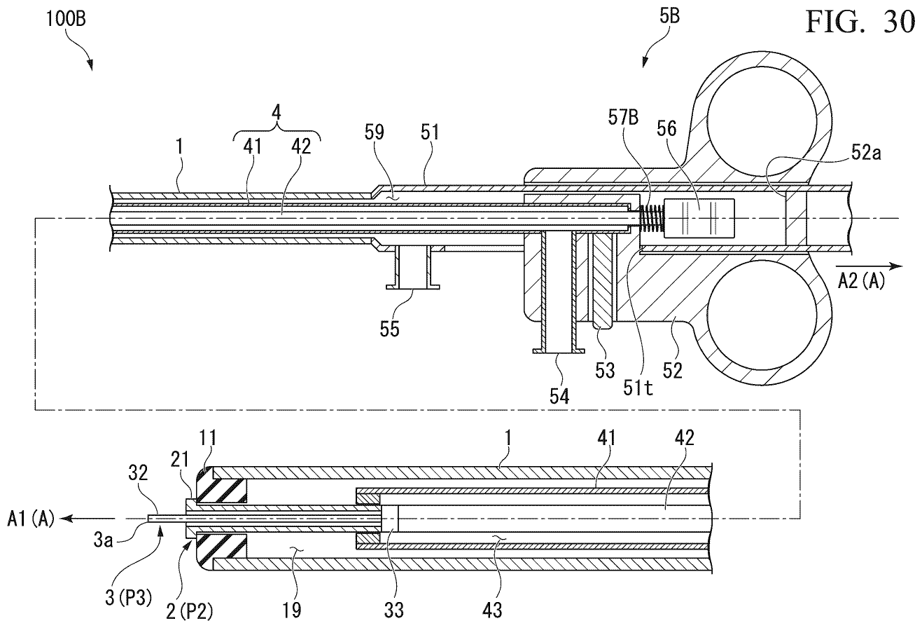

As shown in FIG. 30, the surgeon advances the lever 56 relative to the slider 52 so that the rod 3 advances relative to the knife 2. The rod 3 is positioned at the third position P3, and the distal end portion 32 protrudes from the distal end opening 23a to the distal end side A1. When the surgeon releases the lever 56, the lever 56 moves to the proximal end side A2 due to the elastic force of the elastic body 57B. As a result, as shown in FIG. 29, the distal end 3a of the rod 3 moves to the fourth position P4 to be accommodated in the internal space 43 of the inner tube 41.

According to the treatment instrument 100B of this embodiment, the knife 2 and the rod 3 can be easily operated according to the treatment situation, such as incision and dissection treatment and local injection treatment. The surgeon does not need to operate the lever 56 retracted, and when the surgeon releases the lever 56, the rod 3 is automatically stored in the internal space 43 of the inner tube 41, and hence the procedure is simplified. Further, it is possible to prevent the surgeon from unintentionally operating the knife 2 with the rod 3 protruding. Since the knife 2 and the rod 3 are relatively advanceable and retractable in the longitudinal axis direction A, the stroke amount of the lever 56 relative to the slider 52 can be reduced.

Although the second embodiment of the present disclosure has been described above in detail with reference to the drawings, the specific configuration is not limited to this embodiment, and design changes and the like are also included within the scope of the present disclosure. Further, the components shown in the embodiments and modified examples can be appropriately configured in combination.

### Third Embodiment

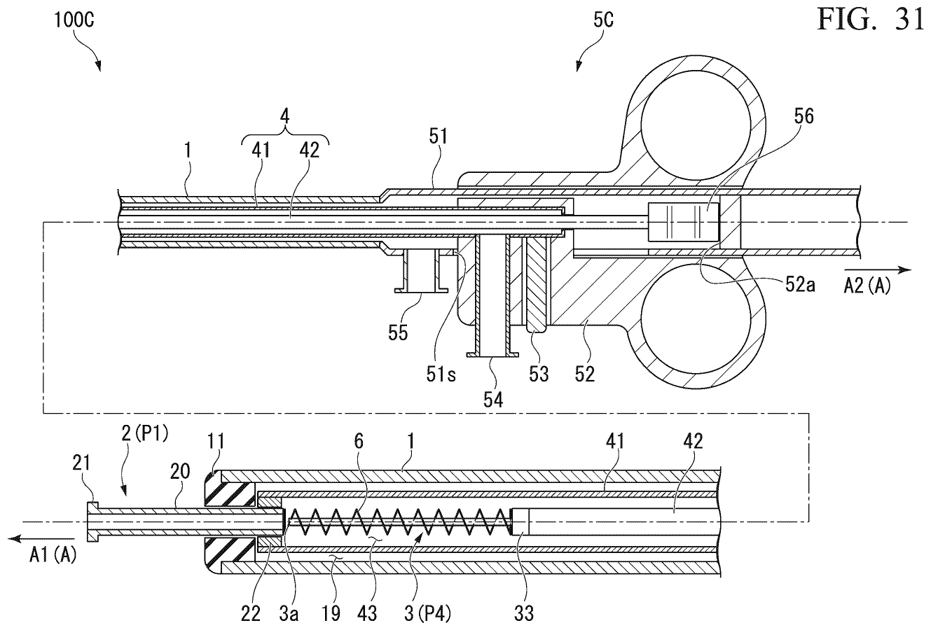

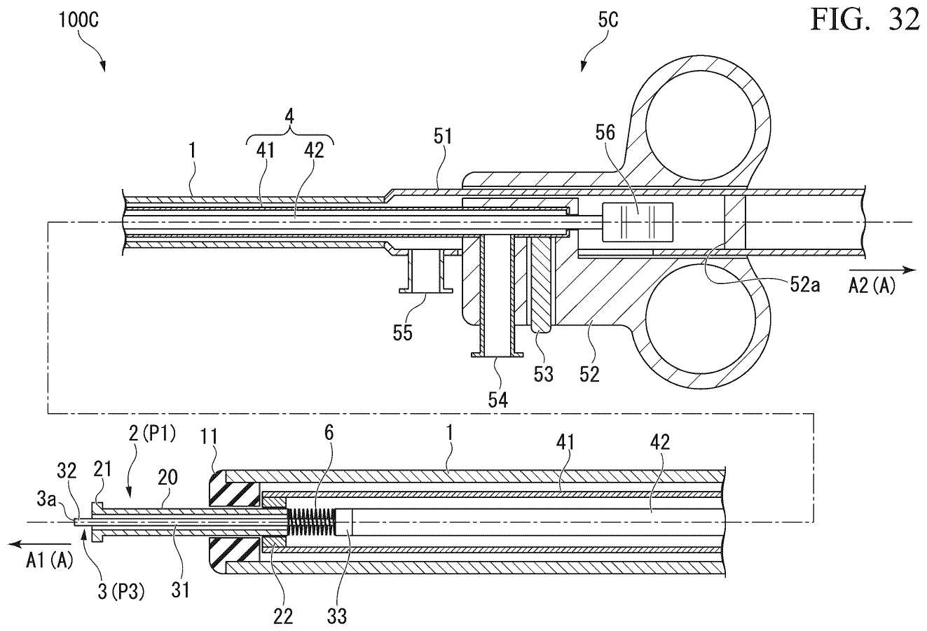

A treatment instrument 100C according to a third embodiment of the present disclosure will be described with reference to FIGS. 31 and 32. In the following description, the same components as those already described will be denoted by the same reference numerals and the description thereof will be omitted.

FIGS. 31 and 32 are diagrams illustrating the operation of the treatment instrument 100C.

The treatment instrument (endoscopic treatment instrument) 100C includes the outer sheath 1, the knife 2, the rod 3, the operation member 4, an operation portion 5C, and the elastic body 6.

The knife 2 is similar to the first embodiment, and includes the knife body 20, the flange 21, and the first connector 22. The knife body 20 and the flange 21 have the first pipeline 23 extending along the longitudinal direction A. The first connector (butting member) 22 connects the knife body 20 and the inner tube 41. The first connector 22 is not insertable through the first through hole 12 in the longitudinal direction A. The first pipeline 23 communicates with the internal space 43 formed in the inner tube 41.

The rod 3 is similar to that of the first embodiment, and has the main body portion 31, the distal end portion 32, and the second connector 33. The rod 3 is inserted through the first pipeline 23 of the knife 2 along the longitudinal direction A, and is capable of protruding and retracting from the distal end opening 23a toward the distal end side A1. The second connector 33 connects the rod 3 and the operation wire 42. The second connector 33 is not insertable through the first pipeline 23 in the longitudinal direction A.

The operation portion 5C has the handle body 51, the slider 52, the power supply plug 53, the liquid delivery port 54, the suction port 55, and the lever 56. The operation portion 5C does not have the elastic body.

The elastic body 6 is located in the internal space 43 of the inner tube 41, and is located between the proximal end 20b of the knife body 20 and the second connector 33 in the longitudinal axis direction A. The elastic body 6 biases the rod 3 toward the proximal end side A2 so that the distal end 3a of the rod 3 is accommodated in the internal space 43 of the inner tube 41. That is, as in the first embodiment, the distal end 3a of the rod 3 is located inside the inner tube 41 while the elastic body 6 returns to an original shape, and when the rod 3 advances against the elastic force of the elastic body 6, the rod 3 is biased by the elastic body 6 toward the proximal end side A2. Furthermore, the distal end 3a of the rod 3 is located on the proximal end side A2 in relation to the distal end of the outer sheath 1 while the elastic body 6 returns to an original shape. Specifically, the elastic body 6 may be a spring, or may be rubber instead of a spring.

As shown in FIG. 31, the surgeon advances the slider 52 relative to the handle body 51 so that the knife 2 advances relative to the outer sheath 1. The knife 2 is positioned at the first position P1. Since the elastic body 6 biases the second connector 33 toward the proximal end side A2, the distal end 3a of the rod 3 moves to the fourth position P4 to be accommodated in the internal space 43 of the inner tube 41. At this time, the distal end 3a of the rod 3 is disposed on the proximal end side A2 in relation to the distal end of the outer sheath 1.

When the slider 52 advances relative to the handle body 51, the lever 56 engages with the first receiving surface 52a formed on the proximal end of the slider 52 and advances together with the slider 52 while being pressed by the first receiving surface 52a. Therefore, the relative positional relationship between the knife 2 and the rod 3 in the longitudinal axis direction A is maintained.

As shown in FIG. 32, the surgeon advances the lever 56 relative to the slider 52 so that the rod 3 advances relative to the knife 2. The rod 3 is positioned at the third position P3, and the distal end portion 32 protrudes from the distal end opening 23a to the distal end side A1. When the surgeon releases the lever 56, the rod 3 moves to the proximal end side A2 due to the elastic force of the elastic body 6. As a result, as shown in FIG. 31, the distal end 3a of the rod 3 moves to the fourth position P4 to be accommodated in the internal space 43 of the inner tube 41.

According to the treatment instrument 100C of this embodiment, the knife 2 and the rod 3 can be easily operated according to the treatment situation, such as incision and dissection treatment and local injection treatment. Further, it is possible to prevent the surgeon from unintentionally operating the knife 2 with the rod 3 protruding. Compared to a case in which the elastic body 57 is provided in the operation portion 5, the treatment instrument 100C is less susceptible to the influence of the curved shape of the insertion portion 202 of the endoscope 200 in terms of the stroke amount of the rod 3, and the amount of the rod 3 puncturing the tissue can be easily controlled.

Although the third embodiment of the present disclosure has been described above in detail with reference to the drawings, the specific configuration is not limited to this embodiment, and design changes and the like are also included within the scope of the present disclosure. Further, the components shown in the above-described embodiments and modified examples can be appropriately configured in combination.

### Modified Example 3-1

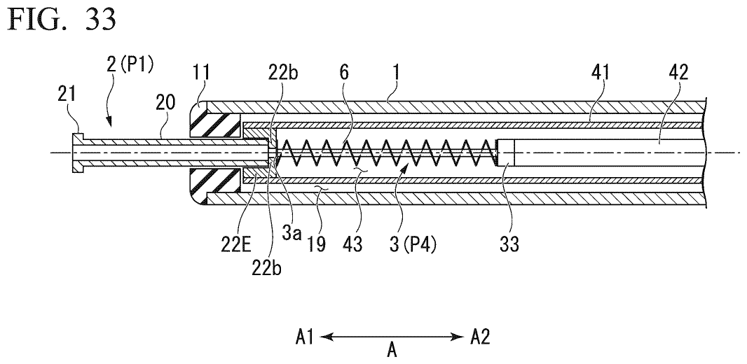

FIG. 33 is a diagram showing a first connector 22E which is a modified example of the first connector 22. The first connector 22E has a reduced diameter portion 22b that is located on the proximal end side A2 in relation to the proximal end 20b of the knife body 20. The elastic body 6 is located in the internal space 43 of the inner tube 41, and is located between the first connector 22E and the second connector 33 in the longitudinal axis direction A.

### Modified Example 3-1

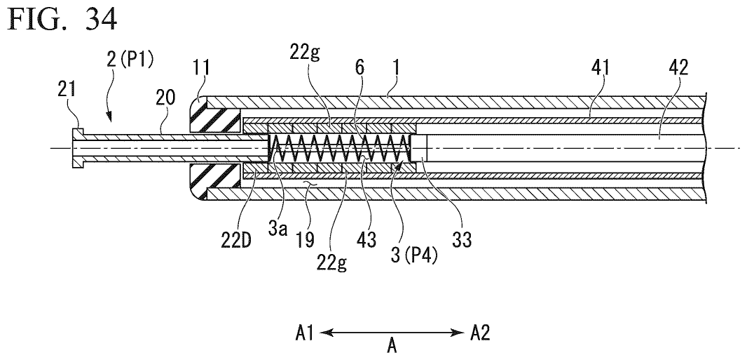

FIG. 34 is a diagram showing a first connector 22D which is a modified example of the first connector 22. The first connector 22D has a guide pipeline 22g extending toward the proximal end side A2. The guide pipeline 22g is a pipeline that accommodates the elastic body 6. The elastic body 6 accommodated in the guide pipeline 22g is guided by the guide pipeline 22g, and can expand and contract smoothly so that the distal end 3a of the rod 3 is not caught by the wire of the elastic body 6. Furthermore, a part of the second connector 33 may enter the guide pipeline 22g of the first connector 22D while the elastic body 6 is restored. Furthermore, the distal end of the operation wire 42 may enter the guide pipeline 22g of the first connector 22D while the elastic body 6 is restored. According to this configuration, since the first connector 22D and the second connector 33 are less likely to get caught, the rod 3 can be smoothly operated relative to the knife body 20.

### Other Modifications

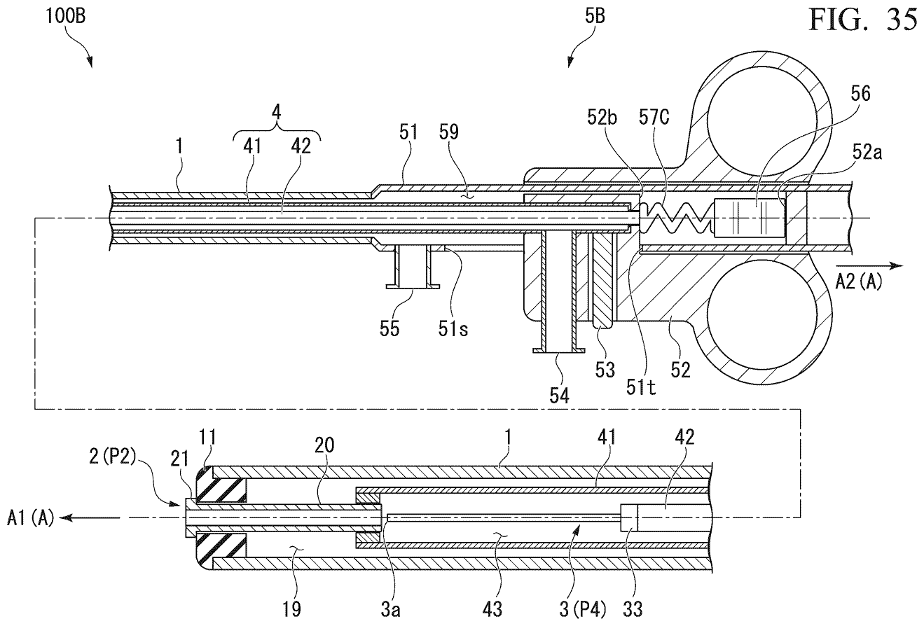

FIG. 35 is a diagram showing an elastic body 57C, which is a modification of elastic body 57B. The elastic body 57C is formed by forming the distal end of lever 56 into a spring shape. In other words, the elastic body 57C and the lever 56 are molded as one piece. The lever 56 and the elastic body 57C have the same effect as the lever 56 and the elastic body 57B, which are separate bodies. A configuration in which multiple parts are molded as one piece, such as the elastic body 57B and the lever 56 being molded as one piece, can be applied to all embodiments and modifications.

The present disclosure can be applied to an endoscopic treatment instrument having a water delivery function.

What is claimed is:

1. An endoscopic treatment instrument comprising:
a sheath;
an electrode which is disposed on a distal end side of the sheath;
a rod, a distal end of the rod capable of being protruded and retracted in the electrode; and
an elastic body which biases a distal end of the rod so that the distal end of the rod is accommodated in more proximal side than a distal end of the electrode.

2. The endoscopic treatment instrument according to claim 1, further comprising:
a handle body which is provided in a proximal end portion of the sheath;
a wire which is connected to a proximal end portion of the rod; and
a first slider which operates the rod,
wherein the elastic body is connected to the wire via the first slider.

3. The endoscopic treatment instrument according to claim 1, further comprising:
an inner tube which extends into the sheath;
a first connector which connects the electrode and the inner tube;
a wire which extends into the inner tube; and
a second connector which connects the rod and the wire,
wherein the second connector comes into contact with the first connector to restrict the protrusion amount of the rod from the electrode.

4. The endoscopic treatment instrument according to claim 1, further comprising:
a handle body which is provided in a proximal end portion of the sheath;
a wire which is connected to a proximal end portion of the rod; and
a first slider which is connected to the wire,
wherein the elastic body is located between the first slider and the handle body.

5. The endoscopic treatment instrument according to claim 4, further comprising:
an inner tube which extends into the sheath; and
a second slider which is connected to a proximal end portion of the inner tube,
wherein the second slider is operable to advance and retract relative to the first slider.

6. The endoscopic treatment instrument according to claim 1, further comprising:
a handle body which is provided in a proximal end portion of the sheath; and
a first slider which operates the rod,
wherein the elastic body is located between the first slider and the handle body.

7. The endoscopic treatment instrument according to claim 6, further comprising:
a second slider which operates the electrode,
wherein the second slider is operable to advance and retract relative to the first slider.

8. The endoscopic treatment instrument according to claim 5,
wherein a distal end of the rod is accommodated in the inner tube not to protrude from a distal end of the electrode while the second slider advances to the maximum extent and the first slider does not advance.

9. The endoscopic treatment instrument according to claim 7, further comprising:
an inner tube which extends into the sheath and is connected to the electrode,
wherein a distal end of the rod does not protrude from a distal end of the electrode and is accommodated in the inner tube while the second slider advances to the maximum extent and the first slider does not advance.

10. The endoscopic treatment instrument according to claim 1,
wherein the elastic body is elastically deformed when a distal end of the rod protrudes from a distal end of the electrode, and
wherein the elastic body returns to an original shape when a distal end of the rod is accommodated in the electrode.

11. The endoscopic treatment instrument according to claim 10,
wherein a distal end of the rod is accommodated in the electrode and a distal end of the rod is located on a proximal end side in relation to a distal end of the sheath while the elastic body returns to an original shape.

12. The endoscopic treatment instrument according to claim 1,
wherein a distal end of the rod is disposed on a proximal end side in relation to a distal end of the electrode retracted to the maximum extent while the elastic body returns to an original shape.

13. The endoscopic treatment instrument according to claim 1, further comprising:
an inner tube which extends into the sheath;
a first connector which connects the electrode and the inner tube;
a wire which extends into the inner tube; and
a second connector which connects the rod and the wire,
wherein the elastic body is disposed between the first connector and the second connector.

14. The endoscopic treatment instrument according to claim 1, further comprising:
a handle body which is provided in a proximal end portion of the sheath;
a wire which is connected to a proximal end portion of the rod;
an inner tube which extends into the sheath;
a first slider which is connected to the wire; and
a second slider which is connected to the inner tube,
wherein the elastic body is located between the first slider and the second slider.

15. The endoscopic treatment instrument according to claim 14,
wherein the first slider engages with the second slider and advances along a longitudinal axis together with the second slider.

16. The endoscopic treatment instrument according to claim 3,
wherein the elastic body is disposed between the first connector and the second connector.

17. The endoscopic treatment tool according to claim 1, wherein
when the distal end of the rod protrudes from the distal end of the electrode, the elastic body elastically deforms, and
when the distal end of the rod is accommodated in more proximal side than the distal end of the electrode, the elastic body returns to an original shape.

18. An endoscopic treatment instrument comprising:
a sheath;
an electrode which is disposed on a distal end side of the sheath;
a rod which is disposed to be advanceable and retractable relative to the electrode; and
an elastic body which biases a distal end of the rod to move toward a proximal end side beyond a distal end of the sheath.

19. The endoscopic treatment instrument according to claim 18, further comprising:
a handle body which is provided in a proximal end portion of the sheath;
a wire which is connected to a proximal end portion of the rod; and
a first slider which operates the rod,
wherein the elastic body is connected to the wire through the first slider.

20. The endoscopic treatment instrument according to claim 18, further comprising:
an inner tube which extends into the sheath;
a first connector which connects the electrode and the inner tube;
a wire which extends into the inner tube; and
a second connector which connects the rod and the wire,
wherein the second connector comes into contact with the first connector to restrict a protrusion amount of the rod from the electrode.

21. An endoscopic treatment instrument comprising:
a tube;
an electrode which is connected to a distal end of the tube; and
a rod which is disposed at least partially inside the electrode,
wherein the endoscopic treatment instrument is configured to bias the rod toward a proximal end of the tube in a state in which a distal end of the rod protrudes from a distal end of the electrode.
